(19) United States
(12) Patent Application Publication
(10) Pub. No.: US 2021/0065248 A1
(43) Pub. Date: Mar. 4, 2021

(54) ADVERTISEMENT PRESENTATION METHOD AND ADVERTISEMENT PRESENTATION SYSTEM

(71) Applicant: MEDIDOC SEARCH INC., MACHIDA-SHI, TOKYO (JP)

(72) Inventor: Shucheng LI, Tokyo (JP)

(21) Appl. No.: 16/963,621

(22) PCT Filed: Jul. 30, 2018
(86) PCT No.: PCT/JP2018/028465

§ 371 Date: Jul. 21, 2020

(30) Foreign Application Priority Data
Jan. 31, 2018 (JP) 2018-014506
Jul. 3, 2018 (JP) 2018-126509

(51) Int. Cl. G06Q 30/02; G16H 50/20; G16H 20/90; G06T 7/00

(52) U.S. Cl. CPC G06Q 30/0271; G06T 7/0012; G16H 20/90; G16H 50/20

(57) ABSTRACT

Provided is an advertisement presentation system capable of presenting an appropriate advertisement to a user. An advertisement presentation system S includes an image recognition unit (20) configured to recognize an image including the teeth and tongue of a user captured by a camera (10) of a portable information terminal (1), a first state recognition unit (21) which recognizes a health state of the teeth based on the image, a second state recognition unit (24) which recognizes a state of the tongue and to recognize a state of physical conditions and body constitutions of the user based on the state, an advertisement acquisition unit (25) which acquires an advertisement based on the state of physical conditions and body constitutions of the user, and an information presentation unit (26) which presents the advertisement together with the health state of the teeth on a touch panel (11) of the portable information terminal (1).

### TECHNICAL FIELD

The present invention relates to an advertisement presentation method for presenting an advertisement suitable for a user based on an acquired image of the user and to an advertisement presentation system.

### BACKGROUND ART

A diagnostic system that captures an image of the oral cavity of a user, diagnoses a state of the oral cavity of the user based on the acquired image, and notifies the diagnostic result to the user has been known. As this kind of diagnostic system, a system of presenting, to a user, advertisements including information of neighboring clinics and information about medical treatments and medical instruments, based on a diagnostic result or a demand from the user has been known (for example, see Patent Literature 1).

### CITATION LIST

### Patent Literature

Patent Literature 1: Japanese Patent Application Laid-Open No. 2009-205330

### SUMMARY OF INVENTION

### Technical Problem

Such a conventional diagnostic system as disclosed in Patent Document 1 is, however, for diagnosing a state of the oral cavity of a user and accordingly presents advertisements only relating to the oral cavity. Hence, the presented advertisements include limited contents, and providers of the system are difficult to ask various business operators for advertisements, unfortunately.

Meanwhile, if presented advertisements include unlimited contents, advertisements completely irrelevant to a user may be presented. As a result, by presenting such an advertisement, a user using the diagnostic system may have stress or uncomfortable feeling.

In consideration of the above disadvantages, an object of the present invention is intended to provide an advertisement presentation method capable of presenting an appropriate advertisement to a user and an advertisement presentation system.

### Solution to Problem

An advertisement presentation method of the present invention includes

an image recognition step of recognizing a first image and a second image from an image of a body of a user by an image recognition unit, the first image being an image of a first region of the body of the user, the second image being an image of a second region of the body of the user,

a first state recognition step of recognizing a first state based on the first image by a first state recognition unit, the first state being a state relating to the first region of the user,

a second state recognition step of recognizing a state relating to the second region based on the second image and recognizing a second state based on the state relating to the second region by a second state recognition unit, the second state being a state relating to a region other than the first region,

an advertisement acquisition step of acquiring an advertisement determined corresponding to the second state by an advertisement acquisition unit, and

an information presentation step of presenting the first state to the user after presentation of the advertisement or simultaneously with presentation of the advertisement by an information presentation unit.

In the description, the “advertisement” includes various advertisements capable of giving information to a user, such as image advertisements, video advertisements, and voice advertisements.

The “recognizing a second state” “based on the state of the second region” means acquiring or calculating a second state based on a health state of the second region (for example, colors or epidermal states) in accordance with a certain rule. For example, this includes a method of acquiring a second state with reference to a data table showing the relation between health states of a second region and second states, a method of calculating a second state based on a health state of the second region by using a certain algorithm, and a method of estimating a second state by presenting an image of the second region to a medical doctor.

Being “determined corresponding to the second state” means being determined with reference to a second state in accordance with a certain rule. For example, this includes a method of acquiring an advertisement based on a content of the second state with reference to a data table showing the relation between contents of a second state and advertisements and a method of acquiring an advertisement based on a content of the second state by using a certain algorithm.

As described above, in the advertisement presentation method of the present invention, a first state pertaining to a first region (i.e., a state of a region that is originally intended to be diagnosed by a user) is recognized, and concurrently a second state relating to a region other than the first region is also recognized based on an image of the second region included in the image captured for the first region. Based on the second state, an advertisement to be presented to a user is then acquired.

Accordingly, an advertisement determined corresponding to the second state is presented to a user together with the first state that relates to the first region and is intended to be diagnosed by the user. In other words, an advertisement presented by the advertisement presentation method to a user is an unconscious advertisement for the user of a system performing the method (i.e., irrelevant to the first region), but is probably useful for the user or probably interests the user (i.e., an appropriate advertisement for a user).

As a result, a system provider using the advertisement presentation method can ask business operators performing businesses relating to a first region and can also ask business operators performing businesses relating to a second region for various advertisements. For users, a presenting advertisement is appropriate for a user, and thus the user is unlikely to have stress or uncomfortable feeling, which could be caused by a presenting advertisement.

In the advertisement presentation method of the present invention, the second state may be a state relating to at least one of a body constitution and a physical condition of the user.

In the description, “states relating to body constitutions and physical conditions” are not limited to a state determined based on western medicine but also include a state relating to body constitutions and physical conditions determined based on traditional Chinese medicine or oriental medicine.

The advertisement presentation method of the present invention preferably includes

a question presentation step of recognizing a state relating to the second region based on the second image and presenting a question based on the state relating to the second region by a question presentation unit, and

an answer recognition step of recognizing an answer to the question by an answer recognition unit, and

in the second state recognition step, the second state recognition unit preferably recognizes the second state based on the second image and the answer.

By only the information obtained based on the second image, the second state is difficult to exactly understand in some cases. Hence, by presenting a question about the second region as above and by referring to the answer, a more appropriate advertisement can be presented to a user.

In the advertisement presentation method of the present invention,

in the first state recognition step, the first state is recognized based on the first image, and severity of the first state is preferably recognized in accordance with a predetermined standard of a first state and severity,

in the second state recognition step, the second state is recognized based on the second image, and severity of the second state is preferably recognized in accordance with a predetermined standard of a second state and severity,

in the advertisement acquisition step, a first advertisement determined corresponding to the first state is preferably acquired, and a second advertisement determined corresponding to the second state is preferably acquired, and

in the information presentation step, the severity of the first state is preferably compared with the severity of the second state, then depending on a comparison result, presentation periods or a presentation order of the first advertisement and the second advertisement is preferably determined, and the first advertisement and the second advertisement are preferably presented.

In the description, the “severity” is, for example, a value recognized based on a state and is determined, for example, based on the seriousness of a disease. The degree of severity can be appropriately determined by a system designer or the like.

When a plurality of types of advertisements are presented and the presentation periods or the presentation order of the advertisements is determined depending on severity (i.e., usefulness for a user) as described above, the presented advertisements can contain more appropriate contents for a user.

In the advertisement presentation method of the present invention,

the first region is preferably a tooth and a gum of the user,

the second region is preferably a region including at least one of a lip, a skin around a lip, and a tongue of the user, and

the advertisement is preferably an advertisement for a Chinese herbal medicine relating to the second state.

In the description, the “skin around lips” means the skin of a region that can be captured together with teeth and gums, such as a jaw, cheeks, a neck, ears, and a nose.

With such a constitution, the first region and the second region can be naturally captured when an image is captured, and thus a user using a system performing the advertisement presentation method of the present invention is more unlikely to have stress or uncomfortable feeling.

The combination of body regions of a user for the advertisement presentation method of the present invention may be any regions that can be simultaneously captured by an image acquisition unit such as a camera. Examples of the region include, in addition to the above regions, eyes and the skin around eyes, eyes and the skin of a forehead, hands, nails, hairs, and the skin around ears.

An advertisement presentation system of the present invention includes

an image recognition unit configured to recognize a first image and a second image from an image of a body of a user, the first image being an image of a first region of the body of the user, the second image being an image of a second region of the body of the user,

a first state recognition unit configured to recognize a first state based on the first image, the first state being a state relating to the first region of the user,

a second state recognition unit configured to recognize a second state based on the second image, the second state being a state relating to a region other than the first region,

an advertisement acquisition unit configured to acquire an advertisement determined corresponding to the second state, and

an information presentation unit configured to present the first state to the user after presentation of the advertisement or simultaneously with presentation of the advertisement.

### BRIEF DESCRIPTION OF DRAWINGS

FIG. 1 is a schematic view showing a schematic configuration of an advertisement presentation system pertaining to an embodiment.

FIG. 2 is a block diagram showing a configuration of the advertisement presentation system in FIG. 1.

FIG. 3 is a flowchart showing processing performed by the advertisement presentation system in FIG. 1.

### DESCRIPTION OF EMBODIMENTS

An advertisement presentation system S pertaining to an embodiment will now be described with reference to drawings. The advertisement presentation system S of the embodiment uses an advertisement presentation system of the present invention as a system for diagnosing the teeth and gums of a user (examining a health state) and for presenting an advertisement for teeth and gums and an advertisement for a Chinese herbal medicine that seems to have an improvement effect on body constitutions and physical conditions of the user.

In other words, the advertisement presentation system S is for acquiring advertisements that are probably useful for a user or probably interest a user (i.e., appropriate advertisements) from a plurality of advertisements provided from business operators intended to show advertisements for teeth and gums (for example, business operators dealing in commercial products affecting teeth and gums, such as toothbrushes) and business operators intended to show advertisements for Chinese herbal medicines and for presenting the acquired advertisements at appropriate timing together with a diagnostic result of the teeth of the user.

The advertisement presentation method and the advertisement presentation system of the present invention are for presenting health information and advertisements based on a plurality of sites that can be simultaneously captured, for example, by a camera of a portable information terminal, and thus are applicable not only to a system for presenting advertisements for teeth and gums and advertisements for Chinese herbal medicines.

Hence, examples of the body region in the advertisement presentation method and the advertisement presentation system of the present invention include, in addition to teeth, lips and the skin around lips, eyes and the skin around eyes, the skin of a forehead, hands, nails, hairs, and the skin around ears. Advertisements other than the advertisement for a region to be diagnosed are not limited to the advertisements for Chinese herbal medicines and may be any advertisement determined corresponding to a state relating to at least one of body constitutions and physical conditions of a user (second state described later).

For example, while the state of hairs is diagnosed, the state of a simultaneously captured skin may be recognized, then the type of stress on a user may be supposed based on the recognized state of the skin, and an advertisement for a fragrance product effective on the stress (specifically, a bath additive or an aromatic oil) may be presented.

For example, the system can be used as a system for diagnosing the teeth and gums of a user (examining a health state) and for presenting an advertisement for teeth and gums and an advertisement for lips or the skin around lips (specifically, the skin of a region that can be captured by a camera 10 of a portable information terminal 1 described later together with teeth and gums, such as a jaw, cheeks, a neck, ears, and a nose) (for example, an advertisement for skin care products including cosmetics).

The “advertisement” in the embodiment is an image or a video, but the “advertisement” in the present invention includes various advertisements capable of giving information to a user, such as voice advertisements, in addition to image advertisements and video advertisements.

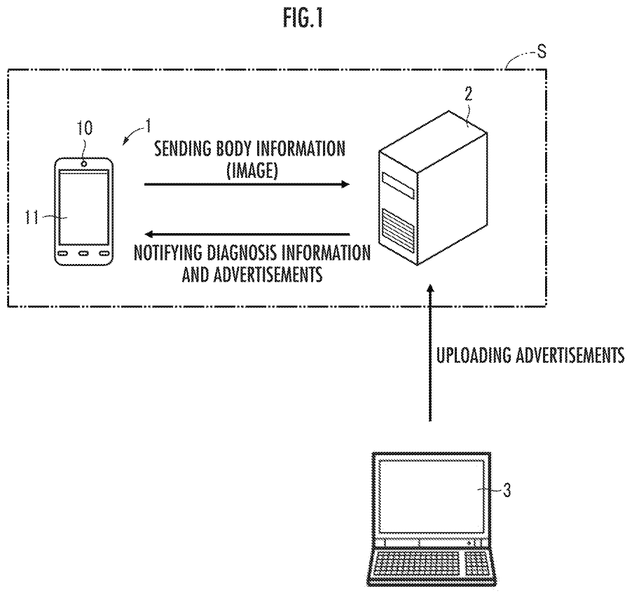

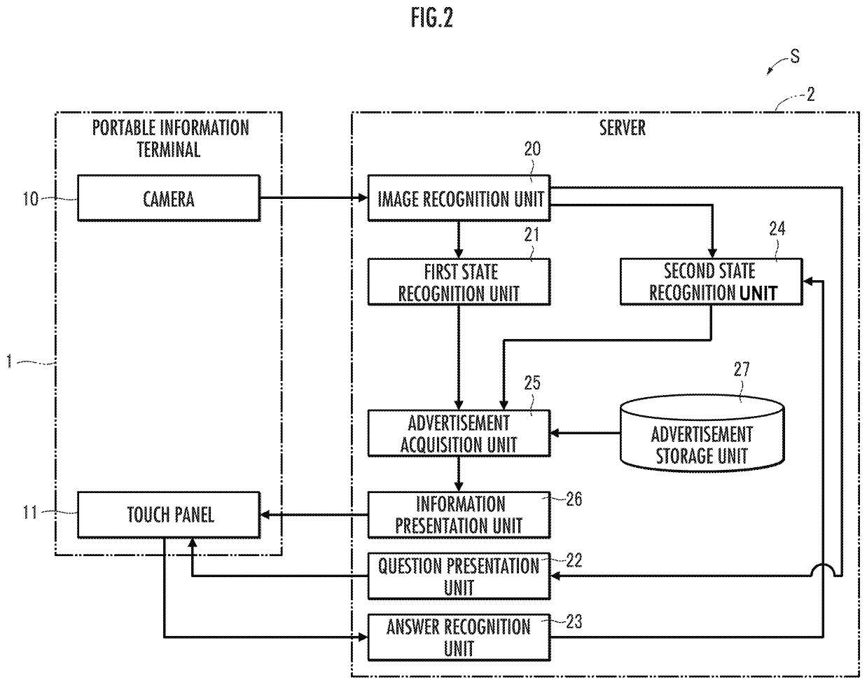

First, with reference to FIG. 1 and FIG. 2, the constitution of the advertisement presentation system S will be described.

As shown in FIG. 1, the advertisement presentation system S includes a portable information terminal 1 (information processing terminal) such as a smartphone held by a user or the like of the system and a server 2 provided by a system provider such as an advertising agency. The portable information terminal 1 and the server 2 are configured to be information-communicably connected together through the Internet connection or the like, and the server 2 and a business operator terminal 3 are configured to be information-communicably connected together through the Internet connection or the like.

The portable information terminal 1 includes a camera 10 (image acquisition unit) for acquiring an image and a touch panel 11 (input-output unit) for presenting the questions and answers described later, a first state, advertisements, and the like to a user and for receiving inputs from the user.

The camera 10 is configured to capture an image of the teeth and gums (first region) together with the lips, the skin around the lips, and the tongue (second region) of a user and to acquire a still image containing them together as a two-dimensional image.

The image acquired by the image acquisition unit of the present invention can be any image from which a first image as an image of the first region and a second image as an image of the second region can be recognized by an image recognition unit 20 described later. For example, the image is not necessarily a two-dimensional image but may be a three-dimensional image. When the image recognition unit can cut a still image from a video and recognize the still image, an image acquisition unit capable of recording videos can be used.

In place of the touch panel 11 that is an input-output unit including an output unit and an output unit, an output unit and an output unit may be used separately. Specifically, a display may be used as the output unit, and a keyboard, a microphone, or the like may be used as the input unit.

As shown in FIG. 2, the server 2 includes, as an installed hardware configuration or functions performed by programs, the image recognition unit 20, a first state recognition unit 21, a question presentation unit 22, an answer recognition unit 23, a second state recognition unit 24, an advertisement acquisition unit 25, an information presentation unit 26, and an advertisement storage unit 27.

The image recognition unit 20 recognizes, based on an image acquired by the camera 10, a first image as an image of the teeth and gums (first region) of the body of a user and a second image as an image of the lips, the skin around the lips, and the tongue (second region).

The first state recognition unit 21 recognizes, based on the first image recognized by the image recognition unit 20, a state of the teeth and gums of a user and recognizes a health state that can be estimated from the state and relates to the teeth and gums (first state) and the severity thereof.

The question presentation unit 22 recognizes, based on the second image recognized by the image recognition unit 20, a state of the lips, the skin around the lips, and the tongue of a user (state relating to the second region). Based on the recognized state, a question to be presented to a user is then determined, and the determined question is presented through the touch panel 11 to the user.

In the embodiment, a method for determining a question can be appropriately selected by a system designer. Examples of the method include a method of determining a question with reference to a predetermined data table showing the relation between recognized states and questions and a method of determining a question based on a recognized state by using a certain algorithm.

The contents of a question can also be appropriately selected by a system designer. For example, when a system is such an advertisement presentation system S of the embodiment as to present an advertisement for a Chinese herbal medicine that seems to have an improvement effect on body constitutions and physical conditions of a user, the contents of a question can be determined from the viewpoint of traditional Chinese medicine or oriental medicine.

Specifically, for example, the skin state of a forehead (black or not), the skin state under eyes (black or not), the skin state of both sides of a face (hollowed, black, or not), the state of a tongue (white or not), the state of lips (purple or not), the state of nails (uneven or not), or the state of hands (joints are curved or not) can be recognized based on a second image.

Depending on these states (states relating to the second region), the liver, the stomach, or the kidneys are suspected to be in a bad condition, but an actual bad condition cannot be determined by only these states. In other words, the body constitutions or physical conditions of a user are difficult to understand exactly.

Hence, when the liver, the stomach, or the kidneys are suspected to be in a bad condition from these states, the question presentation unit 22 is configured to provide a question containing a content capable of acquiring information for determining whether the bad condition is true or not.

Specifically, the question to be presented contains the following contents: do you often take raw foods or cold foods?; do you have swollen legs when waking up in the morning?; frequency of drinking; and frequency of sex, for example. Based on answers to these questions, a state of the tongue (smoothness of the surface, thickness), an odor of the oral cavity, an odor of the body, or the like that cannot be recognized from a second image can be supposed.

As described later, the second state recognition unit 24 then recognizes a second state with reference to the information recognized based on a second image and the information recognized based on the answers to questions.

The answer recognition unit 23 recognizes the answer input through the touch panel 11 by a user to a question represented by the question presentation unit 22.

The second state recognition unit 24 recognizes a state of the lips, the skin around the lips, and the tongue of a user (state relating to the second region) based on the second image recognized by the image recognition unit 20 and recognizes a state that can be estimated from the state and the answer recognized by the answer recognition unit 23 and relates to body constitutions and physical conditions of the user (second state) and the severity thereof.

In the embodiment, the “second state” includes, in addition to the state relating to body constitutions and physical conditions of a user, states that can be estimated or recognized from the state of a second region and relate to regions other than a first region of the body of a user, such as the state relating to a second region itself.

For example, in the advertisement presentation system S of the embodiment, the second region is the lips and the skin around the lips of a user, and the second state is a state relating to body constitutions and physical conditions of the user. Alternatively, the second state may be a state of a region of the body of a user other than the teeth (first region), the lips, and the lips (second region), and the state may be estimated based on a state of the lips and the skin around the lips. Specifically, the state may be a state of the tongue, the oral cavity, or the like estimated based on a state of the lips.

In the advertisement presentation system S, the first state recognition unit 21 and the second state recognition unit 24 recognize the first state and the second state with reference to a comparison result between a previously obtained sample image and the image recognized by the image recognition unit 20.

In the present embodiment, the “state relating to body constitutions and physical conditions” includes, in addition to states based on Western medicine, states relating to body constitutions and physical conditions determined based on traditional Chinese medicine or oriental medicine.

In the present invention, the first state and the second state are not necessarily recognized with reference to the comparison result of images, but may be recognized based on an image recognized by the image recognition unit. For example, the system may be configured to typically recognize a state by comparison of images and, only when a state is difficult to recognize, to send an image to a medical doctor for diagnosis.

The advertisement presentation system S of the embodiment is configured to present a question to a user and to refer to the answer thereto when a second state is recognized, in order to present a more appropriate advertisement to a user. The advertisement presentation system of the present invention is not necessarily limited to such a constitution. For example, when a second state can be recognized highly precisely based on an image, the presentation of a question and the recognition of an answer may be skipped. In this case, no question presentation unit or no answer recognition unit may be included.

In the embodiment, the “severity” is, for example, a value recognized based on a state and is determined, for example, based on the seriousness of a disease. The degree of severity can be appropriately determined by a system designer or the like.

The method of determining severity relating to a first state and a second state can also be appropriately selected by a system designer. Examples of the method include a method of determining severity with reference to a data table as the predetermined standard showing the relation between recognized states and severity and a method of determining severity by using a certain algorithm as the predetermined standard based on a recognized state.

The advertisement acquisition unit 25 acquires, based on a first state and a second state recognized by the first state recognition unit 21 and the second state recognition unit 24, an advertisement for teeth and gums (first advertisement) determined corresponding to the first state and an advertisement for a Chinese herbal medicine (second advertisement) determined corresponding to the second state.

In the embodiment, being “determined corresponding to a state” means being determined with reference to a first state or a second state in accordance with a certain rule. For example, this includes a method of acquiring an advertisement based on the content of a state with reference to a data table showing the relation between contents of a state and advertisements and a method of acquiring an advertisement based on the content of a state by using a certain algorithm.

The information presentation unit 26 compares the severity of a first state and the severity of a second state and determines, depending on the comparison result, the presentation period of a first advertisement, the presentation period of a second advertisement, the presentation order thereof, and the size of each presentation region. In accordance with the determined contents, the information presentation unit 26 presents these advertisements on the touch panel 11 of the portable information terminal 1 and then presents a first state.

The relation between presentation periods and severity comparison results can be appropriately set. For example, an advertisement relating to a state having a higher severity may be presented for a longer period. An advertisement relating to a state having a higher severity may be presented later. An advertisement relating to a state having a higher severity may be presented repeatedly.

In the advertisement presentation system S of the embodiment, the first state recognition unit 21 and the second state recognition unit 24 recognize, together with the first state and the second state, the corresponding severity. This is because the information presentation unit 26 varies the presentation periods and the presentation order of the respective advertisements presented to a user, depending on the severity (i.e., usefulness for a user).

However, for example, when the presentation periods of advertisements are not variable (for example, when only one advertisement is presented), the presentation periods cannot be varied, and thus the system may not be configured to recognize severity or to compare severity.

As the method of varying periods for presenting such advertisements as a still image that does not change with time, the presenting periods can be simply varied. For such an advertisement as a video advertisement that changes with time, a plurality of advertisements having different time periods are acquired for a single state when advertisements are acquired, and an advertisement having a length corresponding to a determined presentation period may be selected from these advertisements.

The comparison result of severity may be reflected in presenting regions in addition to the presenting periods. Specifically, when a plurality of advertisements are simultaneously presented in sectioned display regions on the touch panel 11, an advertisement relating to a state having a higher severity may be presented in a larger region.

The advertisement storage unit 27 stores advertisements uploaded from the business operator terminal 3 of a business operator. The advertisement storage unit 27 may be provided outside the server 2 (for example, another server). No advertisement storage unit 27 may be included, and the information presentation unit 26 may acquire advertisements from the outside as needed.

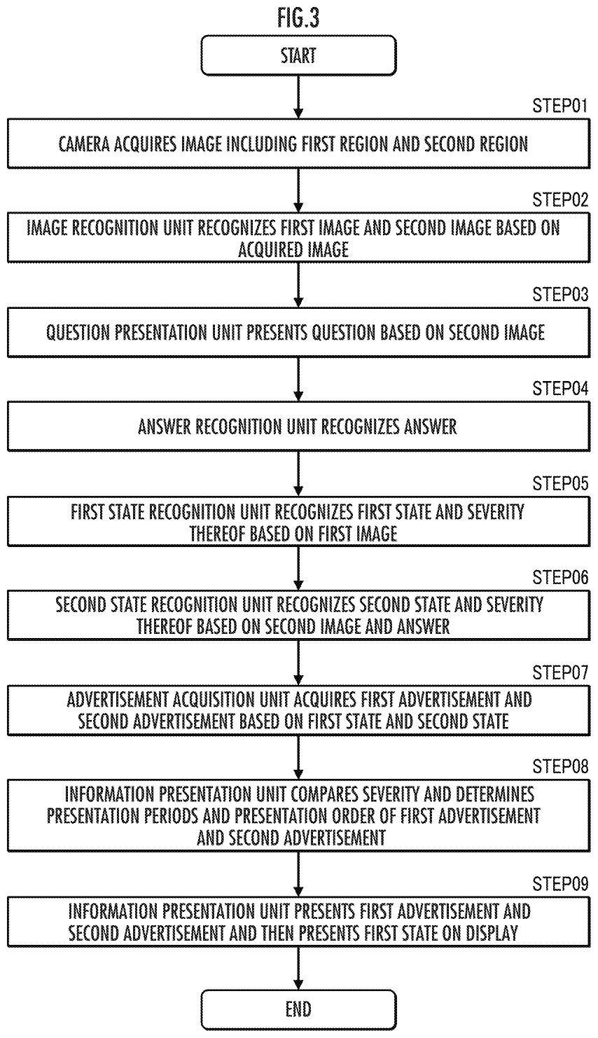

Next, with reference to FIG. 2 and FIG. 3, processing performed by the advertisement presentation system S (the advertisement presentation method of the present invention) will be described. FIG. 3 is a flowchart showing processing performed by the advertisement presentation system S.

In the advertisement presentation system S of the embodiment, a plurality of advertisements capable of being presented are uploaded from the business operator terminal 3 and are stored in the advertisement storage unit 27 before the processing for presenting a diagnostic result and advertisements.

In the processing performed by the advertisement presentation system S, first, the camera 10 of the portable information terminal 1 captures an image of the teeth and gums of a user (first region) together with the lips, the skin around the lips, and the tongue (second region) and acquires a still image including them together as a two-dimensional image (FIG. 3/STEP01).

Next, the image recognition unit 20 of the server 2 recognizes, based on the image acquired by the camera 10, a first image as the image of the teeth and gums (first region) of the body of the user and a second image as the image of the lips, the skin around the lips, and the tongue (second region) (FIG. 3/STEP02 (image recognition step)).

Next, the question presentation unit 22 of the server 2 recognizes, based on the second image recognized by the image recognition unit 20, a state of the lips, the skin around the lips, and the tongue of the user (a state relating to the second region), determines a question based on the state, and presents the question to the user through the touch panel 11 (FIG. 3/STEP03 (question presentation step)).

Next, the answer recognition unit 23 of the server 2 recognizes an answer input by the user through the touch panel 11 to the question presented by the question presentation unit 22 (FIG. 3/STEP04 (answer recognition step)).

Next, the first state recognition unit 21 of the server 2 recognizes, based on the first image recognized by the image recognition unit 20, a state of the teeth and gums of the user and recognizes a health state that can be estimated from the state and relates to the teeth and gums (first state) and the severity thereof (FIG. 3/STEP05 (first state recognition step)).

Next, the second state recognition unit 24 of the server 2 recognizes, based on the second image recognized by the image recognition unit 20, a state of the lips, the skin around the lips, and the tongue of the user (state relating to the second region) and recognizes a state that can be estimated from the state and the answer recognized by the answer recognition unit 23 and relates to body constitutions and physical conditions of the user (second state) and the severity thereof (FIG. 3/STEP06 (second state recognition step)).

Next, the advertisement acquisition unit 25 of the server 2 acquires, based on the first state and the second state recognized by the first state recognition unit 21 and the second state recognition unit 24, an advertisement for teeth and gums determined corresponding to the first state (first advertisement) and an advertisement for a Chinese herbal medicine determined corresponding to the second state (i.e., a Chinese herbal medicine relating to the second state) (second advertisement) (FIG. 3/STEP07 (advertisement acquisition step)).

Next, the information presentation unit 26 of the server 2 compares the severity of the first state and the severity of the second state and determines the presentation periods and the presentation order of the first advertisement and the second advertisement depending on the comparison result (FIG. 3/STEP08).

Next, the information presentation unit 26 presents these advertisements sequentially on the touch panel 11 of the portable information terminal 1 and then presents the first state, and the processing is completed (FIG. 3/STEP09 (information presentation step)).

As described above, in the advertisement presentation system S, the first state pertaining to teeth and gums (first region) (i.e., the state of a region that is originally intended to be diagnosed by a user) is recognized, and the second state relating to body constitutions and physical conditions of the user is also recognized based on an image of the lips, the skin around the lips, and the tongue (second region) included in the image captured for the first region. The second advertisement is then acquired as an advertisement for a Chinese herbal medicine corresponding to the second state.

Accordingly, to a user, the first state as a health state of the teeth and gums intended to be diagnosed by the user as well as an advertisement relating to body constitutions and physical conditions of the user are presented. In other words, the advertisement presented by the advertisement presentation system S to a user is an unconscious advertisement for the user of the system performing the method (i.e., irrelevant to the first region), but is probably useful for the user or probably interests the user.

As a result, a provider of the advertisement presentation system S can ask business operators performing businesses relating to teeth and gums and can also ask business operators performing businesses relating to Chinese herbal medicines for various advertisements. For users, a presenting advertisement is appropriate for a user, and thus the user is unlikely to have stress or uncomfortable feeling, which could be caused by a presenting advertisement.

The embodiments shown in the drawings have been described, but the present invention is not limited to these embodiments.

For example, in the above embodiment, the advertisement presentation system S is configured as a system for diagnosing the teeth and gums of a user and for presenting an advertisement for teeth and gums and an advertisement for a Chinese herbal medicine determined corresponding to a state of the lips, the skin around the lips, and the tongue. This is because an image of lips, the skin around lips, and a tongue is relatively easily acquired together with the capture of an image of teeth and gums.

However, the advertisement presentation method and the advertisement presentation system of the present invention are not limited to such a constitution and can be a method or a system for recognizing, based on a first region of the body of a user and a second region capable of being captured together with the first region, a state relating to either of the body constitutions and the physical conditions of the user and for presenting an advertisement.

For example, the first region may be ears, and the second region may be hairs. Alternatively, the first state may be a state relating to eyes, and the second state may be a state of body constitutions and physical conditions supposed from the skin near the eyes.

In the above embodiment, a plurality of regions of lips, the skin around lips, and a tongue are used as the second region. Alternatively, the second region of the present invention may be only one region. For example, the second region may be only a tongue. When the second region is a plurality of regions as with the above embodiment, the plurality of second regions are not necessarily adjacent regions. For example, a tongue and the skin near eyes may be used as the second region.

In the above embodiment, the first advertisement for teeth and gums pertaining to a first state and the second advertisement for a Chinese herbal medicine pertaining to a second state are presented sequentially.

However, the advertisement presentation method and the advertisement presentation system of the present invention are not limited to such a constitution and can be a method or a system for presenting at least an advertisement pertaining to a second state together with a first state.

For example, as with the advertisement presentation system S of the above embodiment, a system for diagnosing the teeth and for concurrently recognizing physical conditions and body constitutions of a user based on the lips, the skin around the lips, and the tongue may be configured not to present an advertisement pertaining to the teeth and gums (i.e., the first advertisement) but to present only an advertisement for a Chinese herbal medicine relating to physical conditions and body constitutions of the user supposed based on the lips, the skin around the lips, and the tongue (i.e., the second advertisement). A system having such a constitution as above may be configured to present the diagnostic result of the teeth and to concurrently present an advertisement for a Chinese herbal medicine. When two or more second states can be recognized together with a first state, two or more second advertisements may be presented. After presentation of a diagnostic result pertaining to the first region, a second advertisement or a first advertisement and a second advertisement may be presented.

In the above embodiment, the advertisement presentation system S for performing the advertisement presentation method of the present invention includes the server 2 and the portable information terminal 1. However, the advertisement presentation method of the present invention is not necessarily performed only by the system having such a constitution.

For example, a system may include only an information processing terminal that has an image capturing function and is provided at a store front or the like. Alternatively, a system may include only a portable information terminal having processing units and information storage units. Conversely, the processing of recognizing states and acquiring advertisements, which is performed by a single server in the above embodiment, may be performed by separate independent servers.

### DESCRIPTION OF REFERENCE NUMERALS

1 Portable information terminal (information processing terminal)

2 Server

3 Business operator terminal

10 Camera (image acquisition unit)

11 Touch panel (input-output unit)

20 Image recognition unit

21 First state recognition unit

22 Question presentation unit

23 Answer recognition unit

24 Second state recognition unit

25 Advertisement acquisition unit

26 Information presentation unit

27 Advertisement storage unit

1. An advertisement presentation method comprising:
an image recognition step of recognizing a first image and a second image from an image of a body of a user by an image recognition unit, the first image being an image of a first region of the body of the user, the second image being an image of a second region of the body of the user;
a first state recognition step of recognizing a first state based on the first image by a first state recognition unit, the first state being a state relating to the first region of the user;
a second state recognition step of recognizing a state relating to the second region based on the second image and recognizing a second state based on the state relating to the second region by a second state recognition unit, the second state being a state relating to a region other than the first region;
an advertisement acquisition step of acquiring an advertisement determined corresponding to the second state by an advertisement acquisition unit; and
an information presentation step of presenting the first state to the user after presentation of the advertisement or simultaneously with presentation of the advertisement by an information presentation unit.

2. The advertisement presentation method according to claim 1, wherein the second state is a state relating to at least one of a body constitution and a physical condition of the user.

3. The advertisement presentation method according to claim 1, further comprising
a question presentation step of recognizing a state relating to the second region based on the second image and presenting a question based on the state relating to the second region by a question presentation unit; and
an answer recognition step of recognizing an answer to the question by an answer recognition unit, wherein
in the second state recognition step, the second state recognition unit recognizes the second state based on the second image and the answer.

4. The advertisement presentation method according to claim 1, wherein
in the first state recognition step, the first state is recognized based on the first image, and severity of the first state is recognized in accordance with a predetermined standard of a first state and severity,
in the second state recognition step, the second state is recognized based on the second image, and severity of the second state is recognized in accordance with a predetermined standard of a second state and severity,
in the advertisement acquisition step, a first advertisement determined corresponding to the first state is acquired, and a second advertisement determined corresponding to the second state is acquired, and
in the information presentation step, the severity of the first state is compared with the severity of the second state, then depending on a comparison result, presentation periods or a presentation order of the first advertisement and the second advertisement is determined, and the first advertisement and the second advertisement are presented.

5. The advertisement presentation method according to claim 1, wherein
the first region is a tooth and a gum of the user,
the second region is a region including at least one of a lip, a skin around a lip, and a tongue of the user, and
the advertisement is an advertisement for a Chinese herbal medicine relating to the second state.

6. An advertisement presentation system comprising:
an image recognition unit configured to recognize a first image and a second image from an image of a body of a user, the first image being an image of a first region of the body of the user, the second image being an image of a second region of the body of the user;
a first state recognition unit configured to recognize a first state based on the first image, the first state being a state relating to the first region of the user;
a second state recognition unit configured to recognize a second state based on the second image, the second state being a state relating to a region other than the first region;
an advertisement acquisition unit configured to acquire an advertisement determined corresponding to the second state; and
an information presentation unit configured to present the first state to the user after presentation of the advertisement or simultaneously with presentation of the advertisement.
